(19) United States
(12) Patent Application Publication
(10) Pub. No.: US 2010/0049672 A1
(43) Pub. Date: Feb. 25, 2010

(54) Methods for Reducing Sample Size of Clinical Trials

(75) Inventors: Richard C. Straube, Bloomsbury, NJ (US); Ashleigh Palmer, Lebanon, NJ (US)

Correspondence Address: GREENBERG TRAURIG, LLP, ONE INTERNATIONAL PLACE, 20th FL, ATTN: PATENT ADMINISTRATOR, BOSTON, MA, 02110, US

(73) Assignee: Critical Biologics Corporation

(21) Appl. No.: 12/547,069

(22) Filed: Aug. 25, 2009

Related U.S. Application Data
(60) Provisional application No. 61/091,580, filed on Aug. 25, 2008.

(51) Int. Cl. G06Q 90/00; G01N 33/53

(52) U.S. Cl. 705/500; 435/7.1

(57) ABSTRACT

Methods for reducing sample size of clinical trials are disclosed herein. In an embodiment, a method for calculating a sample size for a clinical trial includes choosing values for power, level of significance and size of treatment effect sought for a particular event; selecting a subgroup of people for the clinical trial from a selection of subgroups, the subgroup having a higher value for an event rate than the remaining subgroups; and calculating the sample size for the clinical trial using the values for power, level of significance, size of treatment effect sought and the subgroup event rate, wherein the people of the subgroup have a lower gelsolin concentration than a predetermined baseline value of gelsolin.

### RELATED APPLICATIONS

This application claims the benefit of and priority to U.S. Provisional Application Ser. No. 61/091,580, filed Aug. 25, 2008, the entirety of this application is hereby incorporated herein by reference.

### FIELD

The embodiments disclosed herein relate to methods for reducing sample size of clinical trials, and more particularly to pre-screening patients to select a placebo group of patients that are more likely to experience a poor outcome.

### BACKGROUND

The calculation of the sample size needed for a clinical trial is based on both conventional parameters, such as power, level of significance, and the size of treatment effect sought, as well as on estimated parameters, such as the underlying expected outcome (also referred to as the “event rate”, such as death, need for bypass, etc.) which is measured by the rate in the placebo group. Since sample size calculation for clinical trials is based on the normal deviate curve, increasing the expected event rate, as seen in the placebo group, will lead to a decrease in the sample size needed in order to show a small statistically significant improvement with the experimental therapy. Therefore, if the expected event rate could be increased by pre-screening patients and choosing only those patients that are found to have an increased chance of having a poor event rate, the sample size needed for a particular clinical trial will be less than the sample size needed if patients were not pre-screened.

### SUMMARY

Methods for reducing sample size of clinical trials are disclosed herein.

According to aspects illustrated herein, there is provided a method for stratifying patients into subgroups that includes collecting a body fluid sample from each of the patients; analyzing each of the body fluid samples to determine a concentration of gelsolin; comparing the concentration of gelsolin in each of the body fluid samples to a pre-determined baseline value of gelsolin; and stratifying each of the patients into a subgroup based on the comparison, wherein at least one of the subgroups includes patients that have a lower gelsolin concentration than the pre-determined baseline value, resulting in the patients of the subgroup being more likely to experience a poor outcome. The method may further include analyzing each of the body fluid samples to detect if one of F-actin or actin-gelsolin complexes are present; and stratifying each of the patients into a subgroup based on the detection, wherein at least one of the subgroups includes patients having F-actin or actin-gelsolin complexes, resulting in the patients of the subgroup being more likely to experience a poor outcome.

According to aspects illustrated herein, there is provided a method for calculating a sample size for a clinical trial that includes choosing values for power, level of significance and size of treatment effect sought for a particular event; selecting a subgroup of people for the clinical trial from a selection of subgroups, the subgroup having a higher value for an event rate than the remaining subgroups; and calculating the sample size for the clinical trial using the values for power, level of significance, size of treatment effect sought and the subgroup event rate, wherein the people of the subgroup have a lower gelsolin concentration than a pre-determined baseline value of gelsolin.

According to aspects illustrated herein, there is provided a method for calculating a sample size for a clinical trial that includes choosing values for power, level of significance and size of treatment effect sought for a particular event; selecting a subgroup of people for the clinical trial from a selection of subgroups, the subgroup having a higher value for an event rate than the remaining subgroups; and calculating the sample size for the clinical trial using the values for power, level of significance, size of treatment effect sought and the subgroup event rate, wherein a body fluid sample collected from each of the people of the subgroup have detectable levels of actin-gelsolin complexes.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 is a graph showing the relationship between the placebo event rate and the sample size based on a continuous outcome endpoint. Three different curves are plotted based on the treatment effect sought (i.e., a 25% reduction, a 50% reduction or a 75% reduction).

FIG. 2 is a graph showing the relationship between the placebo event rate and the sample size based on a dichotomous outcome endpoint. Three different curves are plotted based on the treatment effect sought (i.e., a 25% reduction, a 50% reduction or a 75% reduction).

FIG. 3 is a flowchart showing the method steps for stratifying patients into subgroups of the presently disclosed embodiments. The method is based on collecting a body fluid sample from each of the patients and comparing a concentration of gelsolin in the body fluid sample to a pre-determined baseline value of gelsolin.

FIG. 4A shows a schematic illustration of a prior art method for determining the sample size needed for a clinical trial based on both conventional parameters, such as power, level of significance, and the size of treatment effect sought, as well as on estimated parameters, such as the underlying expected event rate as measured in the placebo group.

FIG. 4B shows a schematic illustration of a method for determining the sample size needed for a clinical trial of the presently disclosed embodiments. The method is based on pre-screening patients to design a placebo group that has a high rate of a poor outcome.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Throughout this Application, various publications are referenced The disclosures of these publications are hereby incorporated by reference into this Application in order to more fully describe the state of the art as of the date of the invention described and claimed herein.

Sample size should be planned carefully to ensure that the research time, patient effort and support costs invested in any clinical trial are not wasted. Ideally, clinical trials should be large enough to detect reliably the smallest possible differences in the primary outcome with treatment that are considered clinically worthwhile. It is not uncommon for studies to be underpowered, failing to detect even large treatment effects because of inadequate sample size. Also, it may be considered unethical to recruit patients into a study that does not have a large enough sample size for the trial to deliver meaningful information on the tested intervention.

As used herein, the term “clinical trial” refers to a research study designed to the safety and/or effectiveness of drugs, devices, treatments, or preventive measures in humans or animals. Some clinical trials may last weeks (an acute trial), while others may last months or years (a long-term trial). In an embodiment, the methods disclosed herein are used for calculating a sample size needed to assess the short-term clinical effectiveness of a drug. In an embodiment, the methods disclosed herein are used for calculating a sample size needed to assess the long-term clinical effectiveness of a drug. The methods disclosed herein may be used to calculate the sample size needed for conducting a clinical trial for a disease or disorder selected from the following diseases and disorders, but not limited to, Multiple Sclerosis (MS), Alzheimer's , Rheumatoid Arthritis (RA), Huntington's Chorea (HD), Acute Kidney Failure, Sepsis Syndrome, Acute Respiratory Failure, Acute Lung Injury, Acute Respiratory Distress Syndrome (ARDS), Chronic Kidney Disease, Plasmodium falciparum Malaria, Accelerated Atherosclerosis.

As used herein, the term “sample size” refers to the number of participants in a clinical trial.

As used herein, the terms “placebo group”, “placebo arm”, “control group” and “control arm” refer to the participants in a clinical trial that are given a substance or mock therapy made to look like some form of experimental treatment that has no therapeutic or medicinal qualities.

As used herein, the terms “interventional group”, “interventional arm”, “experimental group” and “experimental arm” refer to the participants in a clinical trial that receive the drug, device, treatment, or intervention under study.

As used herein, the terms “event” and “outcome” refer to the ultimate result of a clinical trial given to patients. Examples of patient-oriented outcomes include, but are not limited to, atherosclerosis, lesions, rheumatoid arthritis score, sepsis, sepsis syndrome, acute respiratory failure, acute lung injury, acute respiratory distress syndrome (ARDS), shock, acute kidney failure, disseminated intravascular coagulation, neutropenia, anemia, increase in length of hospitalization, increase in time on mechanical ventilation, death, overall survival rates, disease-free survival rates, treatment-related morbidity, pro-inflammatory cytokine elevation and elevation of bacterial pro-inflammatory mediators.

As used herein, the term “event rate” refers to the proportion of patients in a group in whom an event is observed.

As used herein, the term “gelsolin” encompasses cytoplasmic gelsolin, plasma gelsolin, as well as fragments thereof. A “fragment” is meant to include any portion of a gelsolin molecule.

The minimum information needed to calculate sample size for a randomized controlled clinical trial in which a specific event is being counted includes the power, the level of significance, the underlying event rate in the population under investigation and the size of the treatment effect sought. The power of a study (denoted by 1−β) is the ability of the study to detect a true difference in outcome between the placebo group and the intervention group. The value for power is usually chosen to be 80%. By definition, a study power set at 80% accepts a likelihood of one in five (that is, 20%) of having the difference between two treatment groups not be statistically significant when one really exists. Thus, the power for large trials is occasionally set at 90% to reduce to 10% the possibility of a so-called “false-negative” result. The chosen level of significance (denoted by a) sets the likelihood of detecting a treatment effect when no effect exists (leading to a so-called “false-positive” result) and defines the threshold “P value”. Results with a P value above the threshold lead to the conclusion that an observed difference may be due to chance alone, while those with a P value below the threshold lead to rejecting chance and concluding that the intervention has a real effect. The level of significance is most commonly set at 5% (that is, P=0.05) or 1% (P=0.01). Two-side statistical tests are most often specified for clinical trials to test both for better and worse outcomes in the intervention group when compared to the placebo group.

Unlike the statistical power and level of significance, which are generally chosen a-priori by researchers based upon convention, the underlying expected event rate (as reflected in the placebo group rate) is typically established by other means, usually from previous studies, including observational cohorts. Usually, investigators start by estimating the event rate in the placebo group. However, estimation of the event rate has mystical overtones, sometimes scant data leads to unreliable estimates. The data often provides the best information available, but may over- or under-estimate event rates, as the data can be from a different time or place, and thus subject to changing and differing background practices. Additionally, trial participants are often “healthy volunteers”, or at least people with stable conditions without other comorbidities, which may further erode the study event rate compared with observed rates in the population. Great care is required in specifying the event rate and, even then, during ongoing trials it may be necessary to adjust the sample size, especially if the overall event rate proves to be unexpectedly low. The effect of treatment in a trial can be expressed as an absolute difference. That is, the difference between the rate of the event in the placebo group and the rate of the event in the intervention group, or as a relative reduction, that is, the proportional change in the event rate with treatment. If the event rate in the placebo group is 6.3% and the event rate in the intervention group is 4.2%, the absolute difference is 2.1%; the relative reduction with intervention is 2.1%/6.3%, or 33%.

Actin is the major protein within many cell types and because of the mass of muscle in humans, may be the most abundant body protein. Cell injury could therefore expose large amounts of actin to the extracellular space, where the ionic conditions favor polymerization of actin into filaments (F-actin), which, in solution, can be many microns in length. Two plasma proteins bind F-actin with high affinity: plasma gelsolin and the vitamin D-binding protein (DVP).

Plasma gelsolin (pGSN) is an about 83 to an about 85 kilo Dalton (kDa) secretory form of cellular gelsolin that circulates in high concentrations in the plasma of all people. The protein is molecularly identical with the cellular form with the addition of a 25 amino acid secretory extension. pGSN consists of six-similar domains that have very different properties. The properties of three of the domains have been discovered: gelsolin segment 1 (G1) and gelsolin segment 4 (G4) binds monomeric actin and gelsolin segment 2 (G2) binds F-actin and tropomyosin. When pGSN is added to F-actin, pGSN severs the filament in a nonproteolytic manner and remains bound to one end of the newly formed filament. If free pGSN molecules are present, the free molecules will sever the filament successively until only 2:1 actin-gelsolin complexes are present, thereby rapidly depolymerizing the filament. Free and complexed (to actin) pGSN molecules differ in functional properties. Although free pGSN can sever filaments, actin-gelsolin complexes cannot. Besides binding to actin and tropomyosin, pGSN appears to importantly bind a series of lipid mediators of inflammation including lysophosphatidic acid, diadenosine phosphate lipopolysaccharide (LPS; endotoxin), lipoteichoic acid (LTA; gram positive bacterial cell wall active lipoprotein), Aβ peptide (a peptide implicated in the pathogenesis of Alzheimer's disease), platelet-activating factor and possibly others. Without the action of the plasma depolymerizing proteins (pGSN and DBP), actin filaments could reach lengths of several microns and might affect blood flow through the microcirculation or cell migration through the extracellular space. The demonstration that long actin filaments can affect fibrin clot formation by inhibiting the lateral association of fibrils into bundles and the abrogation of this effect by shortening the actin filaments with pGSN give some support for the hypothesis that long actin filaments might interfere with normal physiologic processes. Increasing evidence suggests that pGSN, through its ability to bind to the potent early lipid mediators that initiate the inflammatory cascade, also plays a critical role in localizing the inflammatory process to the site of injury and preventing inappropriate systemic inflammation from occurring. It appears that local tissue injury causes the cytoskeleton of damaged cells to be exposed and local, extracellular pGSN avidly binds to the actin causing a local depletion of pGSN. Both exogenous mediators (LPS, LTA) and early endogenous lipid mediators (PAF, LPA, and potential others) are no longer inactivated by binding to the pGSN and can initiate local, beneficial inflammation. Mediators that diffused from the local site of injury to the systemic circulation are rapidly inactivated by binding to the huge, systemic reservoir of pGSN. However, in the uncommon situation in which the extent of tissue is massive, critical depletion of circulating pGSN allows these mediators to “escape” the local site unimpeded and start the process that can lead to inappropriate, potentially life-threatening systemic inflammation such as the sepsis syndrome.

Studies in humans have shown that pGSN levels fall in major inflammatory states including, but not limited to, acute malaria, acute lung injury (ALI), sepsis, critically ill post-surgical patients, major trauma, acute liver injury, and hematopoietic stem cell transplantation. In these studies the degree of decrease in pGSN levels correlates with the extent of tissue injury and the rate of development of disease specific complications such as death in ICU patients and septic patients, the development of idiopathic pneumonia syndrome in stem cell transplant recipients, prolonged mechanical ventilation and ICU stays. Decreases in pGSN levels have also been shown in patients having Alzheimer's , Multiple Sclerosis, Plasmodium falciparum Malaria and Huntington's chorea. Several studies have shown a similar decrease in pGSN levels in animal models including hyperoxia in mice, LPS challenge, oleic acid-induced ALI, cecal ligation/puncture model of sepsis, blast-induced lung injury, and burns. In patients with serious, systemic inflammation, critically depleted pGSN levels have been epidemiologically linked to poor outcome, including the development of the sepsis syndrome and death. pGSN levels differentiate otherwise identically appearing patients that will have higher rates of poor outcomes. Poor outcomes of interest for a clinical trial includes both clinically important events including, but not limited to, the development of sepsis, sepsis syndrome, acute respiratory failure, shock, acute kidney failure, disseminated intravascular coagulation, severe neutropenia, severe anemia and death, as well as biochemical abnormalities including, but not limited to, pro-inflammatory cytokine elevation, elevation of bacterial pro-inflammatory mediators including endotoxin (lipopolysaccharide; LPS) and lysophosphatidic acid (LPA).

By measuring the concentration of gelsolin in body fluid samples taken from patients and/or detecting the presence/absence of F-actin or actin-gelsolin complexes, patients may be stratified into subgroups that are more likely to experience a poor outcome. These measurements may be used as entry criteria for clinical trials to select a patient population in which base outcome event rate (as measured by the rate in the placebo group) will have a higher event rate than it otherwise would have (for example, by the conventional method of estimating the event rate). By increasing the expected event rate, the clinical trial can be completed with fewer patients. This reduction in sample size can be seen regardless of the therapy being examined. The screening of patients for clinical trials by measuring gelsolin levels and/or detecting the presence/absence of F-actin or actin-gelsolin complexes, may be used to identify a subgroup of patients who will experience several-fold higher poor outcome rates than the general patient population.

Clinical trials focus on improving the outcome of patients and use a variety of measures of poor outcome endpoints. Poor outcomes of interest for a clinical trial include, but are not limited to, both clinically important events such as the development of sepsis, the development sepsis syndrome, acute respiratory failure, acute lung injury, acute respiratory distress syndrome (ARDS), shock, acute kidney failure, disseminated intravascular coagulation, neutropenia, anemia, increase in length of hospitalization, increase in time on mechanical ventilation and death, as well as biochemical abnormalities including, but not limited to, pro-inflammatory cytokine elevation and elevation of bacterial pro-inflammatory mediators including endotoxin (lipopolysaccharide; LPS) and lysophosphatidic acid (LPA). The outcome endpoint for a clinical trial is based either on a dichotomous outcome (these are ‘yes’ or ‘no’ outcomes, such as for example, dead or alive, development of acute respiratory distress syndrome (ARDS) or no development, stroke or no stroke, etc) or a continuous outcome (a variable that can theoretically take an unlimited number of values, such as number of hours in the Intensive Care Unit, hours on mechanical ventilation, etc.). The underlying concept of increasing the event rate(s) or event duration resulting in lower patient numbers needed for statistical demonstration of benefit for these two types of endpoints are identical, although the mathematical calculations are different and are presented separately. The statistical literature has extensive descriptions for modifying the basic equations given below to account for specific theoretical conditions. Although use of these modifications will give slight numeric differences in the final calculations, the same fundamental results apply. Pre-screening patients based on gelsolin levels or gelsolin levels and the presence/absence of F-actin or actin-gelsolin complexes can select a patient population with increased expected event rates/durations and this increase allows the clinical trial to be completed with fewer patients. This reduction in sample size follows a nonlinear, chi distribution, so even relatively small changes in the expected outcome rates as measured in the placebo group can have substantial impact on the number of patients needed to show a statistically significant difference in outcomes with the same relative intervention improvement.

As shown mathematically below, for a clinical trial with a fixed statistical threshold, power, and minimally important intervention effect, the greater the expected event rate as measured in the placebo group, the smaller the sample size needed. This relative decrease in sample size with pre-screening patients with gelsolin and/or actin testing is more important the lower the initial event rate in the placebo group. This is important since most critical care trials involve endpoints that occur in an about 15 to an about 30% range. In such situations, an increase in an event rate of an absolute value of about 5 to about 20%, may have a major impact in the sample size needed to reach statistical significance. This in turn has profound impacts on the cost, feasibility, duration and size of the infrastructure necessary to perform the clinical trial.

### Determining Sample Size Needed for a Clinical Trial Based on a Continuous Outcome Endpoint

Methods to calculate sample size needed to show a statistical difference between treatment groups (placebo group vs. intervention group) are well described and accepted based on estimated differences in the values for a primary outcome endpoint. Assuming that the true difference in the values between the intervention and placebo values is represented by “δ” and the experimentally measured mean difference is D̅= X̅_{placebo}− X̅ᵢₙₜₑᵣᵥₑₙₜᵢₒₙ, where X̅_{placebo} and X̅ᵢₙₜₑᵣᵥₑₙₜᵢₒₙ are the mean values for the placebo and intervention endpoint, respectively. Assuming a normal distribution of the difference around the true population differences, the variance of this difference is σ_{D} giving a standard deviation of σ_{D/}\sqrt{n}. In order to be statistically significant, D̅ must exceed Z_{α}σ_{D/}\sqrt{n}, where Z_{α} is the normal deviate corresponding to the significance level α. The distribution of the power function (1−β) is also a normal deviate that can be symbolized by Z_{β}. D̅ has a mean of ∂ with a standard deviation of σ_{D/}\sqrt{n} and hence the quantity ( D̅−∂)/(σ_{D}/√n) is normally distributed. For a two-tailed test, the probability that D̅ exceeds −β (power−1) is given by equation 1:

( D̅−∂)/(σ_{D}/√n)=−Z_{β}_{₍₁₎} (1)

Solving for n, the sample size can be calculated by equation 2:

n=(Z_{α}+Z_{β})^{Z}σ_{D}²/∂² (2)

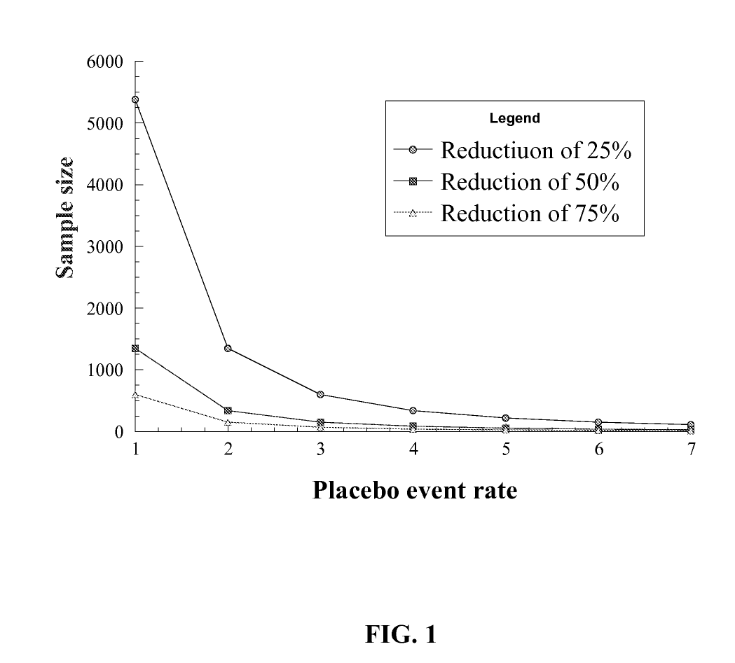

The implication of equation 2 is perhaps best seen by plotting the sample size result for a continuous outcome endpoint against the placebo event rate for a fixed drug effect rate. As shown in FIG. 1, the sample size is plotted for a hypothetical clinical trial in which the α is set at 0.05, the power (1−β) set at 0.90 and the drug effect is set at 25%, 50% or 75% reduction in days in the hospital with a standard deviation for both groups of 2 days. As shown, as the drug effect rate increases the sample size decreases. For each drug effect rate, increasing the event rate in the placebo group decreases the size of the clinical trial to show a statistically significant improvement. Therefore, by selecting a patient population in which the baseline outcome, as measured by the rate in the placebo group, is worse (e.g., increased hospital days, death), the sample size needed is smaller. This indicates that by choosing patients having baseline gelsolin levels that are below a baseline value, the size of the clinical trial may be reduced. Therefore, using a baseline gelsolin level as part of the admission criteria for the placebo group results in a smaller clinical trial.

### Determining Sample Size Needed for a Clinical Trial Based on a Dichotomous Outcome Endpoint

With exactly the same sample results but slightly different underlying derivation, the sample size of a dichotomous outcome endpoint clinical trial yields a result that increasing the event rate in the placebo group of the clinical trial decreases the total sample size of the study. The standard calculation of normal approximation of the binomial equation is shown in equation 3:

n=(Z_{α}+Z_{β})^{Z}(p₁q₁+p₂q₂)/(p₂−p₁)² (3)

Where p₁ is the proportion in the placebo group having the outcome, p₂ is the proportion in the intervention group, Z_{α} is the normal deviate corresponding to the significance level to be used in the test, P′ is the of declaring a significant result, β=2(1−P′), Z_{β} is the normal deviate corresponding to two-tail probability β, q₁ is equal to (1−p₁) and q₂ is equal to (1−p₂).

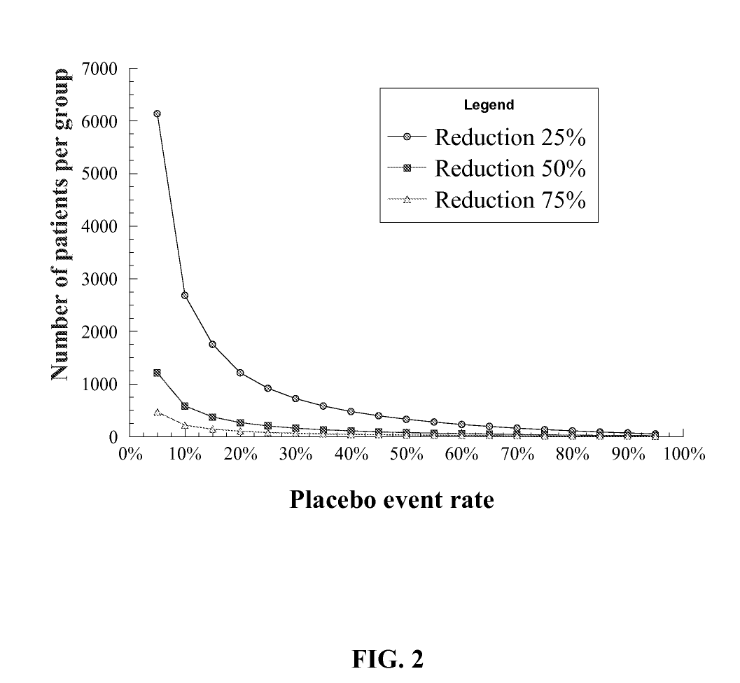

As shown in FIG. 2, the sample size is plotted for a hypothetical clinical trial in which the α is set at 0.05, the power (1−β) set at 0.90 and the drug effect is set at 25%, 50% or 75%. Results indicate that regardless of the drug effect, increasing the event rate in the placebo group decreases the size of the clinical trial needed to show a statistically significant improvement.

According to aspects illustrated herein, a method is provided for stratifying patients into subgroups to create a subgroup of patients that are more likely to experience a poor outcome (for example, death, increased hospital stay, sepsis). The method is based on collecting a body fluid sample from each of the patients and comparing a concentration of gelsolin in each of the samples to a pre-determined baseline value of gelsolin. The subgroup of patients that are more likely to experience a poor outcome have a lower gelsolin concentration than the pre-determined baseline value. The level of the gelsolin for the patient may be obtained by any art recognized method. Typically, the level is determined by measuring the level of the marker in a body fluid, for example, blood, lymph, saliva, urine, cerebrospinal fluid and the like. If the body fluid sample is blood, the blood can be separated into blood cells and plasma. The plasma may further be separated into serum. The level can be determined by ELISA, or immunoassays or other conventional techniques for determining the presence of the marker. Conventional methods include sending a sample(s) of a patient's body fluid to a commercial laboratory for measurement.

The invention also involves comparing the level of gelsolin for the patient with a pre-determined value. The pre-determined value can take a variety of forms. For example, the pre-determined value may be single cut-off value, such as a median or mean. The pre-determined value may be established based upon comparative groups, such as, for example, where the risk in one defined group is double the risk in another defined group. The pre-determined value may be a range, for example, where the tested population is divided equally (or unequally) into groups, such as a low-risk group, a medium-risk group and a high-risk group, or into quartiles, the lowest quartile being subjects with the highest risk and the highest quartile being patients with the lowest risk, or into tertiles the lowest tertile being patients with the highest risk and the highest tertile being patients with the lowest risk. The pre-determined value may depend upon the particular population of patients selected. For example, an apparently healthy population will have a different ‘normal’ range of gelsolin than will a population the subjects of which have had a prior infection or other condition. Accordingly, the pre-determined values selected may take into account the category in which a subject falls.

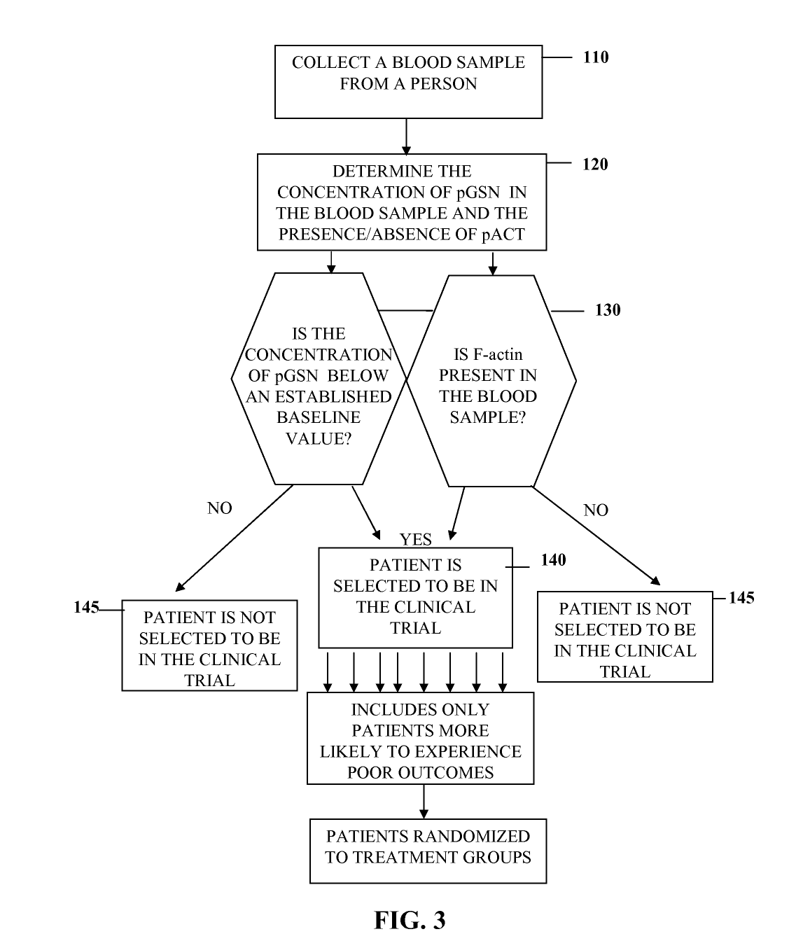

Although the following figures pertain to the collection of a blood sample from a patient for determining a plasma gelsolin concentration, it should be noted that another body fluid can be collected and the concentration of gelsolin in that body fluid can be compared to a pre-determined baseline value of gelsolin. FIG. 3 is a flowchart showing an embodiment of a method for stratifying patients into subgroups. The method pre-screens patients for entry into a clinical trial. The method is based on collecting a blood sample from each of the patients and comparing a concentration of plasma gelsolin in the blood sample to a pre-determined baseline value of plasma gelsolin. Similarly, the same blood sample may be screened for the presence/absence of F-actin or actin-gelsolin complexes. If the patient has a pGSN level that falls below the established baseline value and/or the presence of F-actin or actin-gelsolin complexes is detected, the patient may be selected to be part of the clinical trial. The expected event rate in these patients, which is measured by the event rate in the placebo group, is more likely to experience poor outcomes than patients either that were not pre-screened or did not have the biomarkers (low pGSN or circulating actin). By increasing the expected event rate, as measured by the event rate in the placebo group, the clinical trial can be completed with fewer patients, as will be described and shown in detail below.

Step 110 of the method begins with the collection of a blood sample from an interested patient. An “interested patient” is a patient that would be willing to participate in the clinical trial. Blood may be collected by methods known in the art, and include, for example, using ethylenediaminetetraacetic (ETDA)-containing tubes for collection. Blood samples may be, for example, centrifuged, the plasma component removed and diluted with gel sample buffer, and frozen until needed. In step 120, the concentration of pGSN and/or the presence/absence of F-actin or actin-gelsolin complexes is evaluated. Gelsolin activity may be detected in plasma by techniques including, but not limited to, measuring actin filament nucleating activity (a measure of the total gelsolin concentration), measuring filament-severing activity (free gelsolin activity), and quantitative western blotting. Sepharose beads linked to monoclonal anti-gelsolin antibodies may be used to extract actin-gelsolin complexes from the plasma component of the blood sample. Methods for determining the concentration of pGSN and the presence or absence of F-actin or actin-gelsolin complexes in blood samples are known in the literature (see for example, Janmey P A, Stossel T P: Kinetics of actin monomer exchange at the slow growing ends of actin filaments and their relation to the elongation of filaments shortened by gelsolin. J Muscle Res Cell Motil 1986; 7: 446-454, Mounzer K C, Moncure M, Smith Y R, DiNubile M J: Relationship of admission plasma gelsolin levels to clinical outcomes in patients after major trauma. Am R Respir Crit Care Med 1999; 160: 1673-1681, and Lee P S, Drager L, Stossel T P, Moore F D Jr., Rogers S O: Relationship of plasma gelsolin levels to outcomes in critically ill surgical patients. Ann Surg 2006; 243: 399-403). In step 130, the concentration of pGSN is compared to an established baseline value that has been chosen for the particular clinical trial of interest. Similarly, the presence or absence of F-actin or actin-gelsolin complexes in the sample is determined. If the patient tested has a pGSN concentration that is below the baseline value, and/or the presence of F-actin or actin-gelsolin complexes is determined, the method continues to step 140, and the patient is selected to participate in the clinical trial. If, on the other hand, the pGSN concentration is above the baseline value, and/or there is no detectable F-actin or actin-gelsolin complexes in the sample, the method continues to step 145 and the patient is not selected for the clinical trial. The patients that are selected to be part of the clinical trial are more likely to experience a poor outcome.

The determination of the baseline value for pGSN is arbitrary, and selection requires epidemiologic data with pGSN levels and outcome data of interest. Such data is optimally generated in the patient population of interest but data from other patient populations can be used although the precision of the calculations will be lower than if target-population specific were used. In general, as the baseline pGSN threshold is lowered, the higher the proportion (or numeric measure of outcome) of patients experiencing a poor outcome. However, not all patients presenting with low pGSN will suffer a poor outcome and a few patients with relatively high baseline pGSN will suffer a poor outcome. The number of these “outliers” will depend on the patient population characteristics and the outcome being examined. For each baseline threshold value, the sample size for the potential study can be calculated. Although in general, the smaller the sample size the more “attractive” the study, other considerations such as the inability to analyze the contribution of site effect with few patient per site being enrolled, increased patient heterogeneity with more sites and site for different geographic areas need to be considered. Depending on the constraints of trial being planned (money, number of sites available, number of patients see at each site, amount of drug, and research support including study coordinators, study monitors and project managers), a selection of a baseline threshold value for pGSN level or pGSN level and the presence/absence of F-actin or actin-gelsolin complexes for the specific study is made that provides the most practical patient entry criterion and minimizes the sample size without jeopardizing the statistical analysis.

It has been shown that typical pGSN levels in healthy patients are between about 150 and about 300 mg/L. To date, for patients at risk of developing sepsis syndrome, the critical baseline threshold value for pGSN levels ranges from about 50 to about 100 mg/L, or from about 1400 to about 3000 mU/mL. Similarly, the critical pGSN level appears to range from about 75 to about 140 mg/L or from about 2000 to about 4000 mU/mL for patients recently started on chronic renal hemodialysis. The established baseline value is chosen based on the outcome studied for the clinical trial, for example, if the clinical trial is carried out to determine whether a new drug for treating sepsis syndrome is effective, the baseline value of pGSN may be chosen to be about 100 mg/L. In another embodiment, if the clinical trial is being carried out to determine whether a new procedure is helpful in chronic renal failure, the baseline threshold value of pGSN may be chosen to be about 140 mg/L.

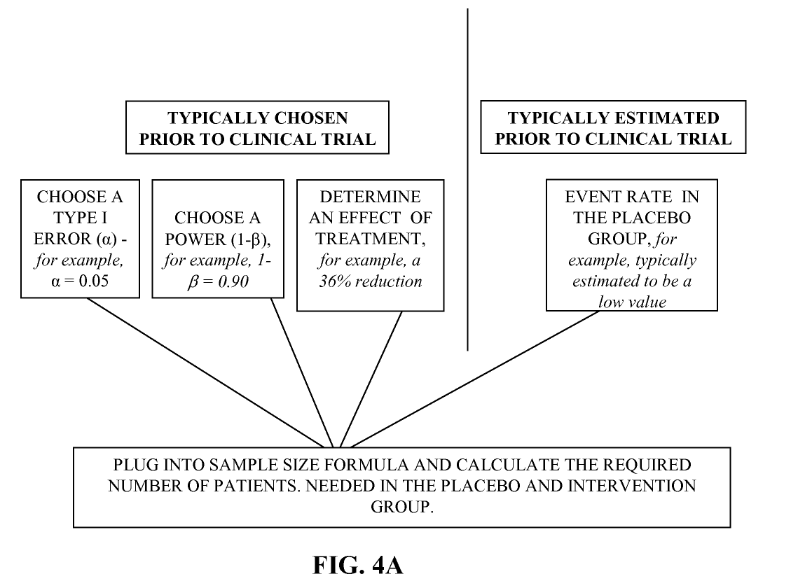

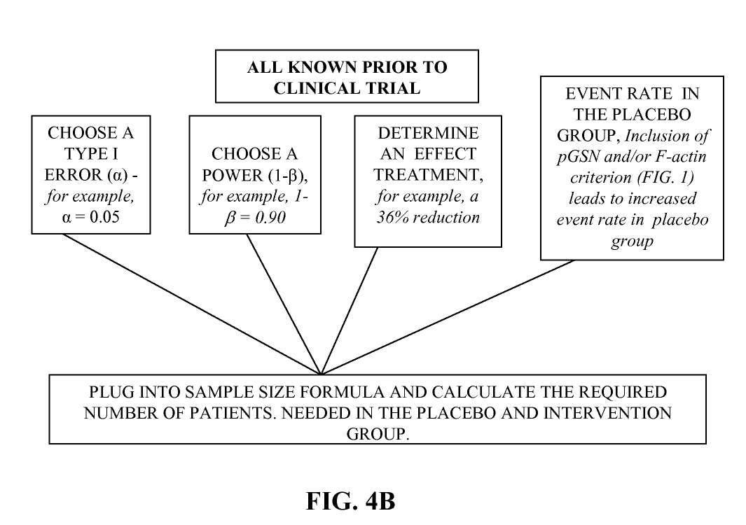

FIG. 4A shows a schematic illustration of a prior art method for determining the sample size needed for a clinical trial. Typically, the power, the level of significance and the effect of treatment are selected a-priori based on conventional parameters. As seen in FIG. 4A, a typical value chosen for the level of significance is 5%, for the power is 90%, and for the effect of treatment is a 36% reduction. As described above, the expected event rate for the population measured by the rate in the placebo group is typically established by other means, usually from previous studies, including observational cohorts. For most clinical trials, the event rate in the placebo group is typically selected to be a low value which leads to a high calculation for the sample size needed.

FIG. 4B shows a schematic illustration of a method for determining the sample size needed for a clinical trial based on the methods of the present disclosure. As described above for FIG. 3, a patient is pre-screened prior to entry into the clinical trial. The patient is pre-screened based on determining the concentration of pGSN in the blood sample and/or the presence/absence of F-actin or actin-gelsolin complexes in the sample. The pGSN level is compared to a baseline value, and the patient is either selected to be in the clinical trial, or alternatively not selected.

A method for stratifying patients into subgroups includes collecting a blood sample from each of the patients; separating each of the blood samples into a blood component and a plasma component; analyzing the plasma component of each of the blood samples to determine a concentration of gelsolin in the plasma; comparing the concentration of gelsolin in the plasma to a pre-determined baseline value of plasma gelsolin; and stratifying each of the patients into a subgroup based on the comparison, wherein at least one of the subgroups includes patients that have a lower plasma gelsolin concentration than the pre-determined baseline value, resulting in the patients of the subgroup being more likely to experience a poor outcome.

A method for calculating a sample size for a clinical trial includes choosing values for power, level of significance and size of treatment effect sought for a particular event; selecting a subgroup of people for a placebo arm of the clinical trial from a selection of subgroups, the subgroup having a higher value for an event rate than the remaining subgroups; and calculating the sample size for the clinical trial using the values for power, level of significance, size of treatment effect sought and the subgroup event rate, wherein the people of the subgroup have a lower plasma gelsolin concentration than a pre-determined baseline value of plasma gelsolin.

A method for calculating a sample size for a clinical trial includes choosing values for power, level of significance and size of treatment effect sought for a particular event; selecting a subgroup of people for the clinical trial from a selection of subgroups, the subgroup having a higher value for an event rate than the remaining subgroups; and calculating the sample size for the clinical trial using the values for power, level of significance, size of treatment effect sought and the subgroup event rate, wherein a blood sample collected from each of the people of the subgroup have detectable levels of actin-gelsolin complexes.

### EXAMPLES

The following examples are illustrative of the benefits of practicing the methods of the presently disclosed embodiments when determining sample size needed for a clinical trial. The methods are based on pre-screening patients and selecting only those patients that have a higher chance of a poor outcome (i.e., a higher event rate). Patients are pre-screened by collecting blood samples and determining the concentration of pGSN levels and/or the presence/absence of F-actin or actin-gelsolin complexes. If the concentration of pGSN is below a baseline value, the patient is selected to be part of the clinical trial.

### Example 1

### Comparison of Sample Size Needed for an Experimental Drug to Reduce Mortality Rate in Patients at Risk of Developing Sepsis Syndrome Admitted to the Hospital

An epidemiology experimental study collected plasma gelsolin (pGSN) levels on patients admitted to the hospital with pneumonia, peritonitis, multiple traumas, Intensive Care Unit admission with a diagnosis of urosepsis, or immune-compromised patients with severe infection. The patients were followed until discharge or death. The overall mortality rate of the patients was about 14%. Among patients with pGSN levels less than about 100 mg/L, the mortality rate was found to be much higher, about 30%.

If a clinical trial was being designed for a theoretical experimental drug therapy to reduce the mortality rate in patients at risk of developing sepsis syndrome admitted to the hospital, the above epidemiology experimental data can be used to calculate the sample size needed. The following assumptions could be made for the desired clinical trial:
The statistical threshold for the trial (α; the probability of avoiding a false positive trial) is set at P=0.05 (5%)
The desired power (1−β; the probability of avoiding a false negative trial) is set at 90%
The effect of treatment sought is set at a 36% reduction
A two-side statistical test is desired

By using a placebo event rate of 14% (the mortality rate seen in all of the patients), the intervention group event rate would be 9% (a relative 36% reduction from the placebo group). Using the above equations,

p₁=0.14 (the placebo group event rate of mortality)

q₁=(1−p₁)=0.86

p₂=0.09 (the intervention group event rate of mortality)

q₂=(1−p₂)=0.91

The normal deviates can be calculated or obtained from published tables:

Z_{α}=Z_{0.05}=1.958, two tailed

Z_{β}=Z_{0.10}=1.282

As discussed above, the standard normal approximation calculation for sample size is:

n=(Z_{α}+Z_{β})^{Z}(p₁q₁+p₂q₂)/(p₂−p₁)²

Substituting the values above into the equation, gives:

n=[(1.958×1.282)²]×[(0.140×0.860)+(0.090×0.910)]/[(0.090−0.140)²]

n=[(3.240)²]×[(0.120)+(0.082)]/[(−0.050)²]

n=10.498×0.202/0.0025

n=849

Thus, a total of 849 patients per group would be needed. In a two group study (drug and placebo, for example), the total sample size would be 1698 patients.

If the same patient entry criteria were used but only patients with pGSN levels less than about 100 mg/L were enrolled, the epidemiology study shows that mortality event rate would be approximately 30%. Using this value as the expected placebo event rate and using the intervention group event rate as 19% (the same relative 36% reduction with treatment), the same sample size calculation is as follows:

p₁=0.30 (the placebo group event rate of mortality)

q₁=(1−p₁)=0.70

p₂=0.19 (the drug treated group event rate of mortality)

q₂=(1−p₂)=0.81

Using the same statistical assumption as before, the normal deviates are:

Z_{α}=Z_{0.05}=1.958, two tailed

Z_{β}=Z_{0.10}=1.282

As before, the standard normal approximation calculation for sample size is:

n=(Z_{α}+Z_{β})^{Z}(p₁q₁+p₂q₂)/(p₂−p₁)²

Substituting the values above into the equation, gives:

n=[(1.958×1.282)²]×[(0.300×0.700)+(0.190×0.810)]/[(0.190−0.300)²]

n=[(3.240)²]×[(0.210)+(0.154)]/[(−0.110)²]

n=10.498×0.364/0.012

n=316

Thus, the number of patients is calculated to be 316 patients per group, or 632 patients in a two-armed study. The above example shows that by pre-screening patients and selecting only those patients that have a higher chance of a poor outcome (by collecting blood samples and determining the concentration of pGSN levels and/or the presence/absence of F-actin or actin-gelsolin complexes), the sample size needed for the clinical trial can be reduced by 60%.

### Example 2

### Comparison of Sample Size Needed for an Experimental Drug to Reduce Mortality Rate in Patients Initiating Hemodialysis with Catheters

A case-control study collected plasma gelsolin (pGSN) levels on patients with end-stage renal disease within two weeks of starting hemodialysis using a catheter rather than a vascular stent or arteriovenous fistula. The patients were followed for one year and the mortality rate was assessed. The estimated one year mortality rate was 40%. In the case-control study, the odds ratio for patients dying in the first 365 days after starting hemodialysis who had a pGSN level less than the median and with measurable plasma actin was 25.9 compared to that of the group that had pGSN levels above the median and no detectable plasma actin. In this study, approximately 65% of the patients had detectable circulating actin. This suggested that in the high risk group (low pGSN levels and the presence of actin) the expected mortality rate over the first year was 80%.

If a clinical trial was being designed for a theoretical experimental drug therapy to reduce the mortality rate in patients initiating hemodialysis with catheters, the above experimental data can be used to calculate the sample size needed. The following assumptions could be made for the desired clinical trial:
The statistical threshold for the trial (α; the probability of avoiding a false positive trial) is set at P=0.05 (5%)
The desired power (1=β; the probability of avoiding a false negative trial) is set at 90%
The effect of treatment sought is set at a 25% reduction
A two-side statistical test is desired

By using a placebo event rate of 40% (the mortality rate assumed for all patients), the intervention group event rate would be 30% (a relative 25% reduction from the placebo group). Using the above equations,

p₁=0.40 (the placebo group event rate of mortality)

q₁=(1−p₁)=0.60

p₂=0.30 (the intervention group event rate of mortality)

q₂=(1−p₂)=0.70

As above, the normal deviates are:

Z_{α}=Z_{0.05}=1.958, two tailed

Z_{β=Z}_{0.10}=1.282

Thus:

n=(Z_{α}+Z_{β})^{Z}(p₁q₁+p₂q₂)/(p₂−p₁)²

n=[(1.958×1.282)²]×[(0.400×0.600)+(0.300×0.700)]/[(0.300−0.400)²]

n=[(3.240)²]×[(0.240)+(0.210)]/[(−0.100)²]

n=10.498×0.450/0.010

n=472

Thus, a total of 472 patients per group or a total of 944 patients in a two group study would be needed.

If the same patient entry criteria were used but only patients with pGSN levels less than the group median value (about 141 mg/L) and the presence of circulating F-actin or actin-gelsolin complexes were enrolled, the expected control rate would be 80%. Using this value as the expected placebo event rate and using the intervention group event rate as 60% (the same relative 25% reduction with treatment), the same sample size calculation is as follows:

p₁=0.80 (the placebo group event rate of mortality)

q₁=(1−p₁)=0.20

p₂=0.60 (the intervention group event rate of mortality)

q₂=(1−p₂)=0.40

with:

Z_{α}=Z_{0.05}=1.958, two tailed

Z_{β}=Z_{0.10}=1.282

As before, the standard normal approximation calculation for sample size is:

n=(Z_{α}Z_{β})²(p₁q₁+p₂q₂)/(p₂−p₁)²

n=[(1.958×1.282)²]×[(0.800×0.200)+(0.600×0.400)]/[(0.600−0.800)²]

n=[(3.240)²]×[(0.160)+(0.240)]/[(−0.200)²]

n=10.498×0.400/0.040

n=105

Thus, the number of patients is calculated to be 105 patients per group, or 210 patients in a two-armed study. The above example shows that by pre-screening patients and selecting only those patients that have a higher chance of a poor outcome (by collecting blood samples and determining the concentration of pGSN levels and the presence/absence of F-actin or actin-gelsolin complexes), the sample size needed for the clinical trial can be reduced by 75%.

In an embodiment, a method for stratifying patients into subgroups and storing information relating to the subgroups on a database includes collecting a body fluid sample from each of the patients; analyzing each of the body fluid samples to determine a concentration of gelsolin; comparing the concentration of gelsolin in each of the body fluid samples to a pre-determined baseline value of gelsolin; and stratifying each of the patients into a subgroup based on the comparison, wherein at least one of the subgroups includes patients that have a lower gelsolin concentration than the pre-determined baseline value, resulting in the patients of the subgroup being more likely to experience a poor outcome. The method may further include analyzing each of the body fluid samples to detect if one of F-actin or actin-gelsolin complexes are present; and stratifying each of the patients into a subgroup based on the detection, wherein at least one of the subgroups includes patients having F-actin or actin-gelsolin complexes, resulting in the patients of the subgroup being more likely to experience a poor outcome. The concentration of gelsolin can be determined by analyzing a body fluid sample using, for example, ELISA, or immunoassays or other conventional techniques for determining the presence of gelsolin. In an embodiment, the database is accessible over a network such as the internet.

In an embodiment, a method for calculating on a computer a sample size for a clinical trial includes choosing values from a database for power, level of significance and size of treatment effect sought for a particular event; selecting, on a computer, a subgroup of people for the clinical trial from a selection of subgroups, the subgroup having a higher value for an event rate than the remaining subgroups; and calculating, on a computer, the sample size for the clinical trial using the values for power, level of significance, size of treatment effect sought and the subgroup event rate, wherein the people of the subgroup have a lower gelsolin concentration than a pre-determined baseline value of gelsolin. In an embodiment, the calculated sample size is stored on a database which is accessible over a network such as the internet.

In an embodiment, a method for calculating on a computer a sample size for a clinical trial includes choosing values from a database for power, level of significance and size of treatment effect sought for a particular event; selecting, on a computer, a subgroup of people for the clinical trial from a selection of subgroups, the subgroup having a higher value for an event rate than the remaining subgroups; and calculating, on a computer, the sample size for the clinical trial using the values for power, level of significance, size of treatment effect sought and the subgroup event rate, wherein a body fluid sample collected from each of the people of the subgroup have detectable levels of actin-gelsolin complexes. In an embodiment, the calculated sample size is stored on a database which is accessible over a network such as the internet.

The embodiments described herein may be implemented using any appropriate computer system hardware and/or computer system software. In this regard, those of ordinary skill in the art are well versed in the type of computer hardware that may be used (e.g., a mainframe, a mini-computer, a personal computer (“PC”), a network (e.g., an intranet and/or the internet)), the type of computer programming techniques that may be used (e.g., object oriented programming), and the type of computer programming languages that may be used (e.g., C++, Basic, AJAX, Javascript). The aforementioned examples are illustrative and not restrictive.

For the purposes of this disclosure, a computer readable medium is a medium that stores computer data in machine readable form. By way of example, and not limitation, a computer readable medium can comprise computer storage media as well as communication media, methods or signals. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology; CD-ROM, DVD, or other optical storage; cassettes, tape, disk, or other magnetic storage devices; or any other medium which can be used to tangibly store the desired information and which can be accessed by the computer.

While a number of embodiments of the present invention have been described, it is understood that these embodiments are illustrative only, and not restrictive, and that many modifications may become apparent to those of ordinary skill in the art. For example, certain methods may have been described herein as being “computer implementable” or “computer implemented”. In this regard, it is noted that while such methods can be implemented using a computer, the methods do not necessarily have to be implemented using a computer. Also, to the extent that such methods are implemented using a computer, not every step must necessarily be implemented using a computer. Further still, the various steps may be carried out in any desired order (and any desired steps may be added and/or any desired steps may be eliminated).

All patents, patent applications, and published references cited herein are hereby incorporated by reference in their entirety. It will be appreciated that several of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

What is claimed is:

1. A method for stratifying patients into subgroups comprising:
collecting a body fluid sample from each of the patients;
analyzing each of the body fluid samples to determine a concentration of gelsolin;
comparing the concentration of gelsolin in each of the body fluid samples to a pre-determined baseline value of gelsolin; and
stratifying each of the patients into a subgroup based on the comparison,
wherein at least one of the subgroups includes patients that have a lower gelsolin concentration than the pre-determined baseline value, resulting in the patients of the subgroup being more likely to experience a poor outcome.

2. The method of claim 1 wherein the body fluid sample is selected from the group consisting of blood, lymph, saliva, urine and cerebrospinal fluid.

3. The method of claim 2 wherein the body fluid sample is blood.

4. The method of claim 1 wherein the gelsolin in the body fluid sample is plasma gelsolin.

5. The method of claim 1 wherein the pre-determined baseline value of gelsolin is about 150 mg/L.

6. The method of claim 1 wherein the pre-determined baseline value of gelsolin is about 100 mg/L.

7. The method of claim 1 wherein the poor outcome is selected from the group consisting of atherosclerosis, lesions, rheumatoid arthritis score, sepsis, sepsis syndrome, acute respiratory failure, acute lung injury, acute respiratory distress syndrome (ARDS), shock, acute kidney failure, disseminated intravascular coagulation, neutropenia, anemia, increase in length of hospitalization, increase in time on mechanical ventilation, death, overall survival rates, disease-free survival rates, treatment-related morbidity, pro-inflammatory cytokine elevation and elevation of bacterial pro-inflammatory mediators.

8. The method of claim 1 further comprising:
analyzing each of the body fluid samples to detect if one of F-actin or actin-gelsolin complexes are present; and
stratifying each of the patients into a subgroup based on the detection,
wherein at least one of the subgroups includes patients having F-actin or actin-gelsolin complexes, resulting in the patients of the subgroup being more likely to experience a poor outcome.

9. The method of claim 8 wherein the concentration of gelsolin is determined by one of measuring actin filament nucleating activity or measuring filament-severing activity.

10. The method of claim 8 wherein the detection of actin-gelsolin complexes is determined by extracting actin-gelsolin complexes using sepharose beads linked to monoclonal anti-gelsolin antibodies.

11. A method for calculating a sample size for a clinical trial comprising:
choosing values for power, level of significance and size of treatment effect sought for a particular event;
selecting a subgroup of people for the clinical trial from a selection of subgroups, the subgroup having a higher value for an event rate than the remaining subgroups; and
calculating the sample size for the clinical trial using the values for power, level of significance, size of treatment effect sought and the subgroup event rate,
wherein the people of the subgroup have a lower gelsolin concentration than a pre-determined baseline value of gelsolin.

12. The method of claim 11 wherein the pre-determined baseline value of gelsolin is about 150 mg/L.

13. The method of claim 11 wherein the pre-determined baseline value of gelsolin is about 100 mg/L.

14. The method of claim 11 wherein the event is selected from the group consisting of atherosclerosis, lesions, rheumatoid arthritis score, sepsis, sepsis syndrome, acute respiratory failure, acute lung injury, acute respiratory distress syndrome (ARDS), shock, acute kidney failure, disseminated intravascular coagulation, neutropenia, anemia, increase in length of hospitalization, increase in time on mechanical ventilation, death, overall survival rates, disease-free survival rates, treatment-related morbidity, pro-inflammatory cytokine elevation and elevation of bacterial pro-inflammatory mediators.

15. The method of claim 11 wherein a blood sample is collected from each of the people of the subgroup, and wherein each of the people of the subgroup have F-actin or actin-gelsolin complexes present in the sample.

16. A method for calculating a sample size for a clinical trial comprising:
choosing values for power, level of significance and size of treatment effect sought for a particular event;
selecting a subgroup of people for the clinical trial from a selection of subgroups, the subgroup having a higher value for an event rate than the remaining subgroups; and
calculating the sample size for the clinical trial using the values for power, level of significance, size of treatment effect sought and the subgroup event rate,
wherein a body fluid sample collected from each of the people of the subgroup have detectable levels of actin-gelsolin complexes.

17. The method of claim 16 wherein the event is selected from the group consisting of atherosclerosis, lesions, rheumatoid arthritis score, sepsis, sepsis syndrome, acute respiratory failure, acute lung injury, acute respiratory distress syndrome (ARDS), shock, acute kidney failure, disseminated intravascular coagulation, neutropenia, anemia, increase in length of hospitalization, increase in time on mechanical ventilation, death, overall survival rates, disease-free survival rates, treatment-related morbidity, pro-inflammatory cytokine elevation and elevation of bacterial pro-inflammatory mediators.

18. The method of claim 16 wherein the body fluid sample collected from each of the people of the subgroup further includes a lower gelsolin concentration than a pre-determined baseline value of gelsolin.

19. The method of claim 18 wherein the pre-determined baseline value of gelsolin is about 150 mg/L.

20. The method of claim 18 wherein the predetermined baseline value of gelsolin is about 100 mg/L.
